SEQUENCE LISTING (13 sequences)

SEQ ID NO: 1 - DNA, 2751 bp, synthetic construct
  source 1..2751: mol_type = other DNA; note = Glycine max (L.) Merr.
atggcgtcgttttccgccgccgtcgctcagttctcccgcgtttcaccttctcacacttcgctccactctcactctcacggcacgctcttccaatctcaatgccgccctttcttcctctcgcgcacttcccattcactccggaagggtcttactttaccacggggaagagaggcaccgagtacatctgtacgtgcttcatttacagatgtttcaccgaatgtgtccttggaggaaaaacaactacccaaaggagaaacttggtctgttcacaaatttggtggaacctgtgtgggaacctctcagagaataaaaaatgttgcggacataattcttaaggatgattcggagaggaaattggtggttgtttctgcaatgtcaaaggtgacagatatgatgtatgaccttatccacaaggctcaatcacgcgatgagtcttatacagctgcattaaatgctgttttggagaagcacagtgcaactgcacatgacatacttgatggagataatcttgctactttcttgtctaaattgcatcatgatattagtaaccttaaggcgatgcttcgtgcaatatacatagctggtcatgcaacagagtcctttacagattttgttgtgggacatggagaattatggtctgctcagatgttgtctctagttattaggaagaatgggactgattgcaaatggatggatacaagggatgtccttatcgtaaatcctactggttctaatcaagttgatcctgactatttggaatctgagcaaagacttgaaaaatggtactctttgaatccatgtaaggtaatcattgccactggattcattgcaagcacacctcaaaacattcctaccacactgaagagagatggaagtgacttctcggcagcaattatgggtgctctatttaaggctcgtcaggtcacaatttggacagatgttgatggtgtgtatagtgcagatcctagaaaagttagtgaggctgtgattttgaagacactgtcttatcaagaggcttgggaaatgtcttattttggtgccaatgtcttgcatccccgcacaattattcctgtgatgcgatatggcatacccattatgataaggaacattttcaacctttctgctcctggaacaaagatctgccatccttctgttaatgatcatgaagatagccagaacctgcaaaattttgtcaaaggatttgcaaccatagacaacttggcacttgtaaacgtcgagggaactggaatggctggtgttccaggtactgccagtgctatttttggtgcagtaaaagatgttggagctaatgttatcatgatatctcaggctagtagtgagcattctgtatgctttgctgtgcccgagaaagaagtaaaagctgttgctgaggcattgcaatctagatttcgtcaagctttggataatgggcgtctttctcaggttgcagtcattccaaattgtagcattctggctgcagttggccagaaaatggcaagcactcctggtgttagtgcctcccttttcaatgcattggctaaggccaatataaatgtccgtgctatagcccaaggttgttctgagtacaatattactgttgttgttaagcgagaggattgtataaaggctttacgagctgtccattccagattttatctctcaagaaccaccatagcaatgggcattattggacctggattaattgggagcacactacttgagcagctaagggatcaggcctcaaccctaaaagaagaattcaacatcgatttgcgtgtaatgggcatacttggttcaaagtcaatgcttcttagtgatgtgggcattgacttagctagatggagagaacttcgagaggaaagaggagaagtggctaatatggaaaaatttgttcaacatgtacatggaaatcattttataccaaacacggcattagtggactgcacagctgactctgtcattgctggctattactatgattggttgcgcaaaggaatacatgtagttactcctaacaagaaggcaaattcaggaccacttgatcagtatttgaagttaagagctcttcaaaggcaatcctatacacattacttctatgaagcaactgtcggagctggtcttccaattgttagcactttacgtggcctccttgaaactggagacaaaatattacaaatcgaaggcatctttagtgggactttgagttacatatttaataactttaaagatggccgggcttttagtgaggtagtttctgaagcaaaggaagcaggttatactgagccagatccaagagatgatctgtctggaacagatgttgccagaaaggttataattcttgctagggagtcgggtttaaagctagaactgtctaatattccagttgaaagccttgtgccagaaccactacgagcttgtgcatcagctcaggagtttatgcaagagctaccaaaatttgatcaggagttcacaaagaaacaagaagatgctgagaatgctggggaagtcttgagatacgttggagtggtggacgtgactaataaaaaaggagtggtagagctgcgaagatacaagaaggatcatccctttgcgcaattgtctgggtcagataacattattgcatttacaacacgaaggtataaggatcagcctctgatagttcgtgggccaggagctggtgctcaagtcaccgctggtggaatatttagtgatattttacgacttgcctcatatcttggtgctccatcgtaa

SEQ ID NO: 2 - protein, 916 aa, synthetic construct
  source 1..916: note = Glycine max (L.) Merr.
MASFSAAVAQFSRVSPSHTSLHSHSHGTLFQSQCRPFFLSRTSHSLRKGLTLPRGREAPSTSVRASFTDVSPNVSLEEKQLPKGETWSVHKFGGTCVGTSQRIKNVADIILKDDSERKLVVVSAMSKVTDMMYDLIHKAQSRDESYTAALNAVLEKHSATAHDILDGDNLATFLSKLHHDISNLKAMLRAIYIAGHATESFTDFVVGHGELWSAQMLSLVIRKNGTDCKWMDTRDVLIVNPTGSNQVDPDYLESEQRLEKWYSLNPCKVIIATGFIASTPQNIPTTLKRDGSDFSAAIMGALFKARQVTIWTDVDGVYSADPRKVSEAVILKTLSYQEAWEMSYFGANVLHPRTIIPVMRYGIPIMIRNIFNLSAPGTKICHPSVNDHEDSQNLQNFVKGFATIDNLALVNVEGTGMAGVPGTASAIFGAVKDVGANVIMISQASSEHSVCFAVPEKEVKAVAEALQSRFRQALDNGRLSQVAVIPNCSILAAVGQKMASTPGVSASLFNALAKANINVRAIAQGCSEYNITVVVKREDCIKALRAVHSRFYLSRTTIAMGIIGPGLIGSTLLEQLRDQASTLKEEFNIDLRVMGILGSKSMLLSDVGIDLARWRELREERGEVANMEKFVQHVHGNHFIPNTALVDCTADSVIAGYYYDWLRKGIHVVTPNKKANSGPLDQYLKLRALQRQSYTHYFYEATVGAGLPIVSTLRGLLETGDKILQIEGIFSGTLSYIFNNFKDGRAFSEVVSEAKEAGYTEPDPRDDLSGTDVARKVIILARESGLKLELSNIPVESLVPEPLRACASAQEFMQELPKFDQEFTKKQEDAENAGEVLRYVGVVDVTNKKGVVELRRYKKDHPFAQLSGSDNIIAFTTRRYKDQPLIVRGPGAGAQVTAGGIFSDILRLASYLGAPS

SEQ ID NO: 3 - DNA, 20 bp, synthetic construct
  source 1..20: mol_type = other DNA
tcgtcgtttcacttcgtttc

SEQ ID NO: 4 - DNA, 20 bp, synthetic construct
  source 1..20: mol_type = other DNA
ttcagataatgcgtgtcgtg

SEQ ID NO: 5 - DNA, 44 bp, synthetic construct
  source 1..44: mol_type = other DNA
cgcgccgggcccaggcctacgcgtatggcgtcgttttccgccgc

SEQ ID NO: 6 - DNA, 39 bp, synthetic construct
  source 1..39: mol_type = other DNA
atcggggaaattcgagctcttacgatggagcaccaagat

SEQ ID NO: 7 - DNA, 22 bp, synthetic construct
  source 1..22: mol_type = other DNA
cctcgttcgaattcgctttttg

SEQ ID NO: 8 - DNA, 22 bp, synthetic construct
  source 1..22: mol_type = other DNA
caactgtcttgtcacttggcat

SEQ ID NO: 9 - DNA, 20 bp, synthetic construct
  source 1..20: mol_type = other DNA
accttctcacacttcgctcc

SEQ ID NO: 10 - DNA, 20 bp, synthetic construct
  source 1..20: mol_type = other DNA
gggtagttgtttttcctcca

SEQ ID NO: 11 - DNA, 40 bp, synthetic construct
  source 1..40: mol_type = other DNA
acaaatctatctctctcgagatggcgtcgttttccgccgc

SEQ ID NO: 12 - DNA, 35 bp, synthetic construct
  source 1..35: mol_type = other DNA
gctcaccatggatcccgatggagcaccaagatatg

SEQ ID NO: 13 - DNA, 23 bp, synthetic construct
  source 1..23: mol_type = other DNA
gctcctacaaatgccatcattgc

(19) United States
(12) Patent Application Publication
(10) Pub. No.: US 2026/0078357 A1
(43) Pub. Date: Mar. 19, 2026

(54) USE OF GLYCINE MAX ASPARTATE KINASE/HOMOSERINE DEHYDROGENASE FAMILY GENE GmAK-HSDH

(71) Applicant: NANJING AGRICULTURAL UNIVERSITY, Nanjing, Jiangsu (CN)

(72) Inventors: Fang HUANG, Nanjing, Jiangsu (CN); Deyue YU, Nanjing, Jiangsu (CN); Shupeng DONG, Nanjing, Jiangsu (CN); Guizhen KAN, Nanjing, Jiangsu (CN); Zhongyi YANG, Nanjing, Jiangsu (CN); Hui WANG, Nanjing, Jiangsu (CN)

(21) Appl. No.: 18/865,711

(22) PCT Filed: Dec. 27, 2023
(86) PCT No.: PCT/CN2023/142483

§ 371 Date: Nov. 14, 2024

(30) Foreign Application Priority Data
Oct. 27, 2023 (CN) 202311404037.6

(51) Int. Cl. C12N 9/12; C12N 9/04; C12N 15/82

(52) U.S. Cl. CPC C12N 9/1217; C12N 9/0006; C12N 15/8243; C12Y 101/01003; C12Y 207/02004

(57) ABSTRACT

Disclosed is use of a Glycine max aspartate kinase/homoserine dehydrogenase family gene GmAK-HSDH. The gene GmAK-HSDH encoding a GmAK-HSDH protein has the nucleotide sequence set forth in SEQ ID NO: 1. A constructed overexpression vector pBA002-GmAK-HSDH is transformed into a recipient material JACK by cotyledonary node transformation.

### CROSS REFERENCE TO RELATED APPLICATION

The present application is a national stage application of International Patent Application No. PCT/CN2023/142483, filed on Dec. 27, 2023, which claims priority to the Chinese Patent Application No. 202311404037.6, filed with the China National Intellectual Property Administration (CNIPA) on Oct. 27, 2023, and entitled “USE OF GLYCINE MAX ASPARTATE KINASE/HOMOSERINE DEHYDROGENASE FAMILY GENE GmAK-HSDH”, which is incorporated herein by reference in its entirety.

### REFERENCE TO SEQUENCE LISTING

A computer readable XML file entitled “Sequence Listing”, that was created on September 14, with a file size of 1,6396 bytes, contains the sequence listing for this application, has been filed with this application, and is hereby incorporated by reference in its entirety.

### TECHNICAL FIELD

The present disclosure belongs to the technical field of plant genetic engineering, and relates to use of a Glycine max aspartate kinase/homoserine dehydrogenase family gene GmAK-HSDH. The present disclosure specifically relates to use of the gene GmAK-HSDH, which is derived from a cultivated soybean plant and encodes two major functional domains of aspartate kinase (AK) and homoserine dehydrogenase (HSDH), in regulating water-soluble protein content and protein content in soybean seeds.

### BACKGROUND

Cultivated soybean (Glycine max (L.) Merr.) is an important seed crop widely distributed around the world, originally domesticated from wild soybean (G. soja Sieb. and Zucc.). As a major oilseed crop, soybean provides valuable vegetable oil and protein for food, feed, and industrial applications. Soy protein is generally easily soluble in water, and only water-soluble protein can be processed and utilized in traditional soy foods making the water-soluble protein content an important indicator for soy food processing.

The first step in the biosynthesis of aspartate-derived amino acids is the conversion of aspartate into aspartate-4-phosphate by monofunctional AKs and bifunctional AK-HSDHs. Subsequently, the aspartate-4-phosphate is converted into aspartate semialdehyde, where a branch point intermediate is used for the biosynthesis of Lys or Met, Thr, and Ile. One of the major branches is the biosynthesis of Met, Thr, and Ile, with a key step being the formation of homologous serine from aspartate semialdehyde catalyzed by bifunctional AK-HSDHs. Higher plants have multiple genes encoding AK and AK-HSDH. For example, a nuclear genome of Arabidopsis thaliana contains three AK-encoding genes (AK1 [At5g13280], AK2 [A5g14060], and AK3 [At3g02020]) and two AK-HSDH-encoding genes (AK-HSDH1 [At1g31230] and AK-HSDH2 [At4g19710]) (Jander and Joshi, 2009). Published microarray data indicate that these five enzymes are expressed in all plant tissues. Transcription of these enzymes is repressed in darkness and other low-glucose conditions by essential leucine zipper transcription factors. Furthermore, both the AKs and AK-HSDHs are subject to feedback inhibition by downstream products. For instance, the activity of the monofunctional AKs is inhibited by Lys feedback, which is mediated by two lysine-binding ACT domains at the C-terminus of the protein. The activity of the AK-HSDHs is inhibited by Thr feedback through two Thr-binding ACT domains in a middle part of the protein. Teresa and Lu, through free and protein-bound amino acid profile analysis, transcript abundance analysis, and activity analysis on a series of monofunctional and bifunctional deletion mutants of the AK and AK-HSDH, ultimately found that a total activity ratio of the AK and HSDH is negatively correlated with a total amount ratio of the Lys to Met, Thr, and Ile. The balance between Lys-sensitive monofunctional AKs and Thr-sensitive bifunctional AK-HSDHs is important for maintaining the levels and ratios of aspartate and derived amino acids thereof.

### SUMMARY

An objective of the present disclosure is to provide high-quality use of a gene GmAK-HSDH encoding two major functional domains of AK and HSDH in genetic engineering. The gene GmAK-HSDH is expressed in all soybean tissues, and subcellular localization shows that a protein encoded by GmAK-HSDH is localized in the chloroplast. Overexpression of the gene GmAK-HSDH in soybean increased the water-soluble protein content and protein content of soybean seeds.

The GmAK-HSDH, as a target gene, is over-expressed in a recipient material through genetic engineering, thereby changing the water-soluble protein content and protein content in soybean seeds.

In this study, the water-soluble protein content of a natural population consisting of 272 cultivars from 25 provinces of three major soybean producing areas in China and from overseas soybean producing areas such as the United States and Brazil, provided by the National Soybean Improvement Center of Nanjing Agricultural University, is measured in three environments. Combining re-sequencing markers for genome-wide association analysis (GWAS), a gene Glyma.08G107800 encoding two major functional domains of AK and HSDH is discovered at a significantly associated SNP block on chromosome 8, and may affect the water-soluble protein content in soybean seeds. AK-HSDH is a bifunctional AK/HSDH as well as one of the important rate-limiting enzymes in the amino acid biosynthesis pathway for aspartate family.

The objective of the disclosure can be achieved by the following technical solution:

The present disclosure provides use of a soybean bifunctional enzyme encoding gene GmAK-HSDH in water-soluble protein content and protein content of genetically engineered soybean seeds, where an encoding region of the soybean bifunctional enzyme encoding gene GmAK-HSDH has the sequence set forth in SEQ ID NO: 1, and an encoded protein has the amino acid sequence set forth in SEQ ID NO: 2.

Overexpression of the gene GmAK-HSDH in soybean increases the water-soluble protein content and protein content of soybean seeds.

### Beneficial Effects

In the present disclosure, GmAK-HSDH encodes the bifunctional AK/HSDH. Tissue expression analysis has revealed that GmAK-HSDH express in all soybean tissues, with higher expression levels in leaves and 35-day-old seeds. Subcellular localization has showed that the GmAK-HSDH protein is localized in chloroplasts. Overexpression of GmAK-HSDH in soybean variety JACK has revealed that transgenic soybean seeds have increased water-soluble protein content and protein content compared with those of the control. Statistical analysis of the water-soluble protein content and protein content of transgenic soybean seeds show that GmAK-HSDH-overexpressed transgenic lines can significantly increase the water-soluble protein content and protein content of soybean seeds. In the present disclosure, the utility of the gene in regulating soybean quality traits is disclosed, which can improve the quality of crops by directed modification of the water-soluble protein content and protein content of crops.

Using a plant overexpression vector pBA002-GmAK-HSDH, GmAK-HSDH can be introduced into the plant body to regulate the quality traits of plant seeds, thereby obtaining transgenic plants.

### BRIEF DESCRIPTION OF THE DRAWINGS

The present disclosure is described in more detail with reference to the accompanying drawings and examples.

FIG. 1 shows cloning of the GmAK-HSDH gene; where primers are designed based on the GmAK-HSDH sequence information predicted by the Phytozome13 website, and PCR amplification is conducted using the leaf cDNA of the cultivated soybean material NJAU_089 as a template to obtain a 2,751 bp DNA fragment; after sequencing analysis, the sequence information of the fragment is consistent with the sequence predicted by the Phytozome website, that is, the 2,751 bp fragment is the GmAK-HSDH gene; Marker: 5 k, from bottom to top, the bands are 300, 500, 800, 1,000, 1,500, 2,000, 3,000, 5,000 bp, respectively.

FIG. 2 shows tissue expression analysis of the GmAK-HSDH gene; where real-time fluorescence quantitative PCR is conducted to study the expression of GmAK-HSDH in different tissues of cultivated soybean material NJAU_089, and the different tissues of cultivated soybean are root, stem, leaf, flower, 7-day-old (7-d) pod, 14-day-old (14-d), 21-day-old (21-d), 28-day-old (28-d), and 35-day-old (35-d) seeds.

FIG. 3 shows subcellular localization of the GmAK-HSDH gene; where GFP represents green fluorescent protein; Chl represents chloroplast autofluorescence; BF represents bright field; Merge represents the superposition of signals from each channel; the scale bar is 50 μm.

FIG. 4 shows the bar strip test of TO-generation overexpressing transgenic plants; where 1 represents JACK, 2 to 4 represent three TO-generation positive individuals, respectively.

FIGS. 5A-5B show the positive identification of T2-generation overexpressing transgenic plants and the relative expression level of GmAK-HSDH; where FIG. 5A indicates PCR detection of the target gene in T2-generation transgenic soybean lines; partial vector sequence and GmAK-HSDH CDS sequence were amplified with specific detection primer pairs; Marker: 2 k, from bottom to top, the bands are 100, 250, 500, 750, 1,000, 2,000 bp, respectively; P: positive plasmid; N: negative control; FIG. 5B shows the detection of an expression level of the GmAK-HSDH gene in different strains; the relative expression level is an expression level relative to the control after normalization by tubulin gene (relative expression level in CK is 1); OE-3, OE-4, and OE-6 represent different transgenic strains, and JACK represents the control recipient material; * indicates a significant difference at the level of 0.01<p<0.05; ** indicates an extremely significant difference at the level of p<0.01.

FIG. 6 shows the statistical analysis of water-soluble protein content in transgenic soybean seeds; where OE-3, OE-4, and OE-6 represent different transgenic strains, and JACK represents the control recipient material; * indicates a significant difference at the level of 0.01<p<0.05; ** indicates an extremely significant difference at the level of p<0.01.

FIG. 7 shows the statistical analysis of protein content in transgenic soybean seeds; where OE-3, OE-4, and OE-6 represent different transgenic strains, and JACK represents the control recipient material; * indicates a significant difference at the level of 0.01<p<0.05; ** indicates an extremely significant difference at the level of p<0.01.

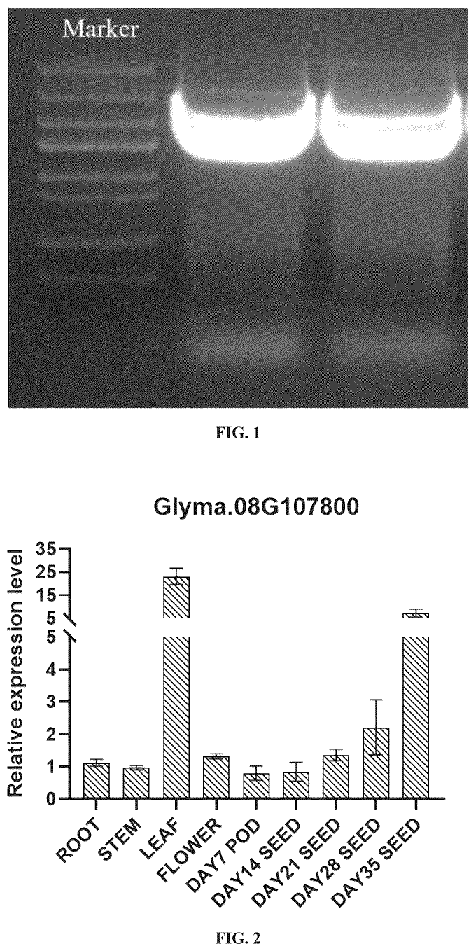

### DETAILED DESCRIPTION OF THE EMBODIMENTS

The present disclosure is further described in detail below in conjunction with specific examples and with reference to data. It should be understood that the examples are provided to merely illustrate the present disclosure and do not limit the scope of the present disclosure in any way. In the following examples, various processes and methods that are not described in detail are conventional methods known in the art. The primers used are all marked when they appear for the first time, and the same primers used thereafter are all the same as those marked for the first time.

### Example 1 Cloning and Identification of Soybean Bifunctional Enzyme Gene GmAK-HSDH

The NJAU_089 experimental material provided by the National Soybean Improvement Center of Nanjing Agricultural University was planted at the NAU Pailou Teaching Research Base of Nanjing Agricultural University in June 2021. PCR amplification was conducted using a leaf cDNA in the seedling stage as a template and GmAK-HSDH-F and GmAK-HSDH-R as primers.
Upstream primer GmAK-HSDH-F: TCGTCGTTTCACTTCGTTTC; (SEQ ID NO: 3)
Downstream primer GmAK-HSDH-R: TTCAGATAATGCGTGTCGTG (SEQ ID NO: 4)

The GmAK-HSDH gene was amplified from a total RNA of soybean leaf organs by RT-PCR. The soybean leaf tissue was crushed in a mortar, added into a 1.5 mL EP tube containing lysis solution, shaken thoroughly, and then transferred to a glass homogenizer. After homogenization, the mixture was transferred to another 1.5 mL EP tube, and total RNA was extracted using a novel plant total RNA extraction kit (TIANGEN) according to instructions. The mass of total RNA was identified by formaldehyde denaturing gel electrophoresis, and the RNA content was determined by spectrophotometer. The total RNA was used as a template to allow reverse transcription according to the instructions of a reverse transcription kit HiScript®IIQ RT SuperMix for qPCR (+gDNA wiper) provided by Vazyme Biotech Co., Ltd to synthesize the first chain of cDNA. PCR amplification was conducted. The PCR reaction system consisted of: 2 μL of cDNA (0.05 μg), 2 μL each of upstream and downstream primers (10 μM), 25 μL of 2×Phanta Max Buffer, 1 μL of dNTP (10 mM), and 1 U of Phanta Max Super-Fidelity DNA polymerase (Vazyme), and supplementing to 50 μL with ultrapure water. The PCR program was conducted on a Bio-RAD PTC200 PCR instrument, including: pre-denaturation at 95° C. for 3 min; 35 cycles of denaturation at 95° C. for 15 s, annealing at 58° C. for 15 s, and extension at 72° C. for 95 s; then extension at 72° C. for 5 min to terminate the reaction, and storage at 4° C. The PCR product was recovered and cloned into a pCE2 TA/Blunt-Zero vector. After sequencing, the cDNA sequence of the soybean GmAK-HSDH gene with a complete coding region, SEQ ID NO: 1, was obtained. The gene had a total length of 2,751 bp, and could encode 916 amino acids set forth in SEQ ID NO: 2.

### Example 2 Expression Characteristics of GmAK-HSDH in Different Organs of Cultivated Soybean

RNA was extracted from roots, stems, leaves, flowers, 7-d pods, and 14-d, 21-d, 28-d, and 35-d seeds of NJAU_089 material and converted into cDNA for RT-PCR analysis.

The extraction of total RNA was the same as that in Example 1. The soybean constitutively expressed gene Tubulin was used as a reference gene, and its amplification primers included: Tubulin forward primer sequence: CCTCGTTCGAATTCGCTTTTTG (SEQ ID NO: 7), Tubulin reverse primer sequence: CAACTGTCTTGTCACTTGGCAT (SEQ ID NO: 8). Real-time fluorescence quantitative PCR was conducted using the cDNA from different soybean tissues or organs as a template. The amplification primers of GmAK-HSDH were GmAK-HSDH-qPCR-F: ACCTTCTCACACTTCGCTCC (SEQ ID NO: 9), GmAK-HSDH-qPCR-R: GGGTAGTTGTTTTTCCTCCA (SEQ ID NO: 10). The analysis of the results (FIG. 2) showed that GmAK-HSDH was expressed in various tissues of soybean throughout the entire growth period, with higher expression levels in leaves and 35-d seeds, and showed an increasing expression trend during seed development, which might indicate that GmAK-HSDH had a certain effect on the development of soybean seeds.

### Example 3 Subcellular Localization of GmAK-HSDH

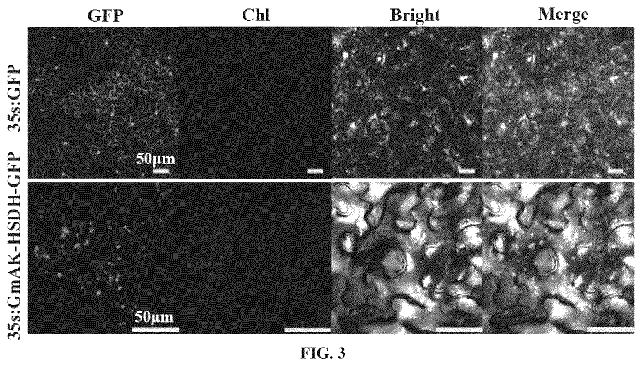

Subcellular localization was conducted using transient expression method in Nicotiana benthamiana, with a vector P2 and primers including: GmAK-HSDH-P2-F: ACAAATCTATCTCTCTCGAGATGGCGTCGTTTTCCGCCGC (SEQ ID NO: 11), GmAK-HSDH-P2-R: GCTCACCATGGATCCCGATGGAGCACCAAGATATG (SEQ ID NO: 12). PCR amplification was conducted, and a target band was cut and recovered after it was qualified correct. The recovered product was ligated to a vector by homologous recombination to construct a subcellular localization vector P2-GmAK-HSDH (with the gene located at the N-terminal of GFP). After the expression of Nicotiana benthamiana, culture was conducted for 48 h in the dark and laser irradiation with a laser confocal microscope (Zeiss, LSM780) was completed to generate a green fluorescent signal to locate the protein, and images were observed and photographed. The results were shown in FIG. 3 to FIG. 5. The transferred empty plasmid was distributed throughout the cells, and GmAK-HSDH: GFP fusion protein was concentrated in the chloroplasts, indicating that GmAK-HSDH might mainly function through chloroplast-rich organs, such as leaves.

### Example 4 Genetic Engineering Use of GmAK-HSDH

1) Construction of Overexpression Vector pBA002-GmAK-HSDH

The vector used for creating the overexpression transgenic soybean was pBA002, and the primers were GmAK-HSDH-pBA002-F: CGCGCCGGGCCCAGGCCTACGCGTATGGCGTCGTTTTCCGCCGC (SEQ ID NO: 5), GmAK-HSDH-pBA002-R: ATCGGGGAAATTCGAGCTCTTACGATGGAGCACCAAGAT (SEQ ID NO: 6). After PCR amplification, gel excision and recovery were conducted after the target band was correct. The gel recovery product was ligated to the vector by homologous recombination, in which the pBA002 vector was double-digested with MluI and SacI restriction endonucleases, and then recombined and ligated. The product, the expression vector pBA002-GmAK-HSDH, was transformed into Escherichia coli DH5a, and the transformation solution was spread on LB solid medium containing 50 mg/L Kana to screen positive clones. After sequencing verification, the plasmid was extracted to obtain the pBA002-GmAK-HSDH plant overexpression vector, which was then transferred into an Agrobacterium tumefaciens strain EHA 105 using a freeze-thaw method.

2) Genetic Transformation of Soybean with Plant Overexpression Vector and Knockout Vector

Transgenic soybean plants were created using the cotyledon node transformation method mediated by Agrobacterium tumefaciens strain EHA 105. The specific creation was as follows:

The soybean seeds that showed no surface defect, had full grains, and exhibited a uniform seed coat color were selected and sterilized in a fume hood. The chemical reaction HCl (concentrated)+NaClO→Cl₂↑+NaOH (a volume ratio of concentrated hydrochloric acid to sodium hypochlorite was about 1:10) occurred to produce chlorine gas for sterilization. In the experiment, 120 mL of NaClO was placed in a conical flask. The soybeans in the culture dish were placed in a dryer, and the conical flask was placed in the center of the dryer and covered with a lid. 15 mL of concentrated HCl was slowly added from a top side of the dryer through a separatory funnel to allow sterilization for 6 h to 7 h.

Seed germination: after sterilization, the seeds were fully blown with chlorine in a clean bench and vertically inserted into a pre-prepared and solidified SG4 germination solid medium such that the medium covered half of the hilum.

Inoculation: about 120 mL of a YEB broth with antibiotics Kan and Rif was added into another conical flask, 1-2 mL of a small amount of bacterial solution was added, and shaken at 28° C. and 200 rpm until OD₆₀₀ reached 0.85-0.9.

Agrobacterium infection: the shaken bacterial solution was centrifuged at 5,000 rpm for 10 min at room temperature, the supernatant was discarded, then a co-culture medium CCM was added into two centrifuge tubes, oscillated and suspended, and adjusted to OD₆₀₀ of 0.5-0.6. 5 days after the soybean seeds germinated, a part of the hypocotyl was removed to retain 5 mm to 10 mm, then the seeds were cut off along the cotyledons and hypocotyl, the true leaves were removed, and then several cuts were slightly made using a knife along a direction of the hypocotyl at the cotyledon node. Treated explants and the bacterial suspension obtained were poured into a sterilized jar, and co-cultivated for 30 min to 40 min at 28° C. and 120 rpm. Finally, the explants were taken out and placed on a filter paper on a CCM solid medium with a cotyledonary node side facing downwards, where 14 explants were placed in each petri dish and cultivated for 5 d at 25° C. in the dark.

Induction of clustered buds: after 5 d of co-cultivation, the explants were sterilized with sterile water and Wash-Liquid, and excessively long hypocotyls were cut off to leave about 5 mm to 10 mm. The expalnts were inserted into SIM solid medium without glufosinate at an angle of 45° with the growth point upward, where 8 explants were placed in each petri dish and cultivated for 14-17 d at 26° C. under light. After 14-17 d, the large buds and part of the hypocotyls were removed, and the explants with clustered buds were transferred to SIM solid medium supplemented with 6 mg/L glufosinate to allow screening culture for 14-17 d.

Elongation: incomplete dead cotyledons, dead leaves, and part of the hypocotyls of the explants were removed, and the resulting explants were transferred into SEM solid medium supplemented with 4 mg/L glufosinate to allow cultivation for 14-17 d. The dead leaves and part of the hypocotyls were removed while the explants were replaced on a new SEM solid medium at a cycle of 14-17 d, with gradually decreasing a glufosinate concentration.

Rooting: when the bud of explant grew to about 6 cm, the bottom of explant was cut off, a cross-shaped wound was made at the bottom of the stem, and the explants were transferred into a rooting medium RM to allow culture for about 10 d, and then induced roots could be seen.

Hardening of seedlings: an appropriate amount of sterile water was poured into the bottle and cultured at 26° C. for about 5 d. Transplanting was conducted when the number and length of roots are appropriate, specifically, the tissue culture seedling was separated from the medium, transferred into a sterilized soil, and cultivated in an artificial incubator (16 h light/8 h dark, 25° C.).

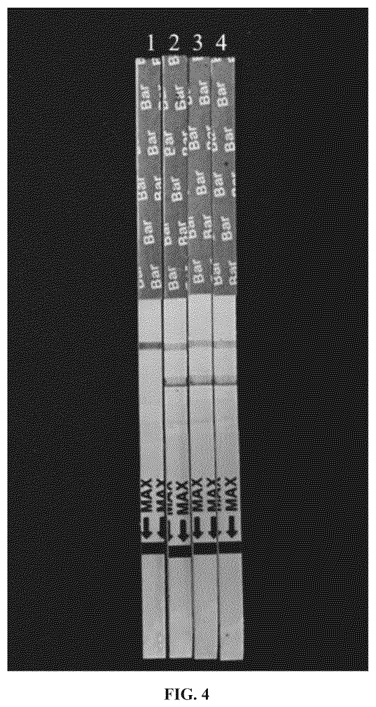

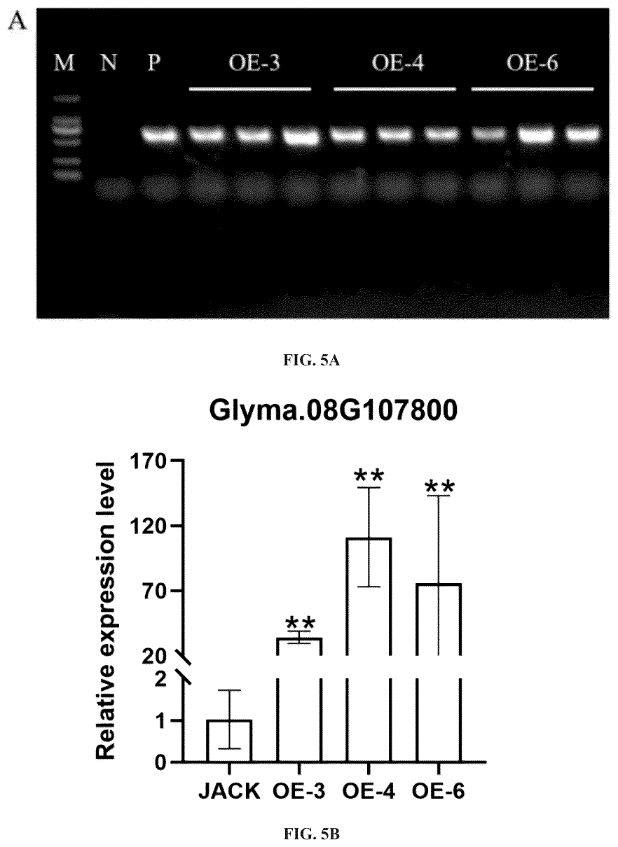

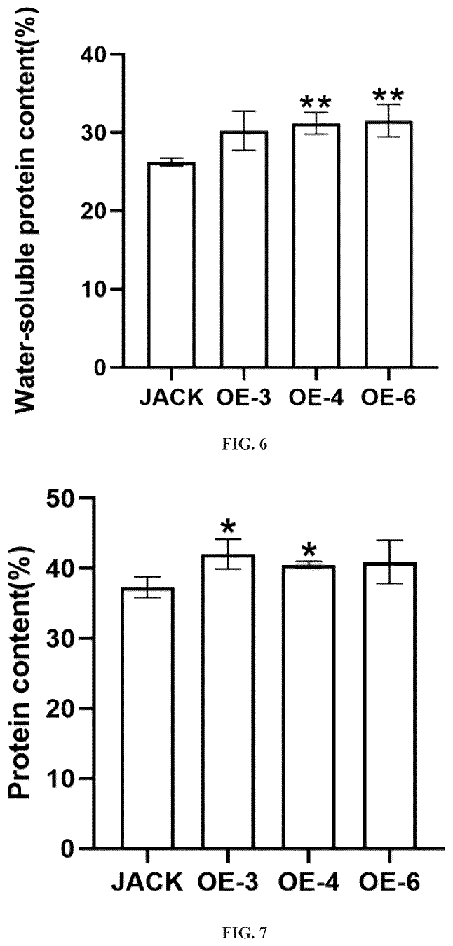

The overexpression vector pBA002-GmAK-HSDH contained the selection marker gene bar in its vector sequence. After the transplanting of TO-generation seedlings, the TO-generation soybean plants were positively detected using the bar rapid detection paper strip (FIG. 4). After the TO-generation seedlings grew new leaves, leaves were collected from different nodes for mixed testing. After adding 500 μL of buffer, the leaves were crushed and mixed evenly, the test strip was inserted vertically into the mixed solution and the results were read after the mixed liquid rose to a certain height of the test strip. If there was only one band above, the sample was negative. If two bands were displayed, it meant the sample was positive, and the bar protein could be detected and the sample plant was considered to be a positive plant. The genomic DNA of TO-generation transgenic plants with bar resistance obtained by preliminary screening was extracted, and PCR identification was conducted using vector and gene-specific primers 35s-F: GCTCCTACAAATGCCATCATTGC (SEQ ID NO: 13) and GmAK-HSDH-qPCR-R: GGGTAGTTGTTTTTCCTCCA (SEQ ID NO: 10). The results showed that the three transgenic lines obtained were all soybean positive plants successfully transformed with the overexpression vector (FIG. 5A). After growth and generation in a laboratory environment, in the T2 generation, real-time fluorescence quantitative PCR detection was conducted using GmAK-HSDH-qPCR-F: ACCTTCTCACACTTCGCTCC (SEQ ID NO: 9) and GmAK-HSDH-qPCR-R: GGGTAGTTGTTTTTCCTCCA (SEQ ID NO: 10) as primers. The results showed that the expression levels of GmAK-HSDH in the transgenic lines OE-3, OE-4, and OE-6 were significantly increased compared with that in the receptor JACK (FIG. 5B).

### 3) Determination of Water-Soluble Protein Content and Protein Content in Transgenic Soybean Seeds

After tissue culture experiments, a total of 3 positive overexpression lines were obtained. After reproduction and generation in the laboratory environment, T2-generation plants were planted in a net house and T3-generation seeds were harvested. The seeds were placed in a 28° C. oven, and the water-soluble protein content and protein content of the seeds were determined using a near-infrared spectrometer one week later. The water-soluble protein content in seeds of the three overexpression lines was higher than that of the receptor JACK, among which OE-4 and OE-6 strains reached extremely significant levels. The results of the determination of seed protein content after maturity showed that the seed protein content of the three overexpression lines also showed an increasing trend compared with JACK, among which OE-3 and OE-4 strains reached extremely significant levels. In summary, the overexpression of GmAK-HSDH can increase the contents of water-soluble protein and protein in soybean seeds, creating germplasm resources for the breeding of high-yield and high-quality new varieties, and providing new ideas for the cultivation of high-quality soybean.

1. (canceled)

2. (canceled)

3. A recombinant expression vector containing a protein encoding gene GmAK-HSDH, wherein the protein encoding gene GmAK-HSDH is aspartate kinase/homoserine dehydrogenase family gene GmAk-HSDG with the nucleotide sequence set forth in SEQ ID NO. 1.

4. The recombinant expression vector according to claim 3, wherein the recombinant expression vector has a plasmid pBA002.

5. A method for increasing water-soluble protein content and protein content in soybean seeds, comprising using the gene GmAK-HSDH according to claim 3.

6. A method for increasing water-soluble protein content and protein content in a soybean seeds, comprising using the recombinant expression vector according to claim 3.

7. The method according to claim 5, wherein the soybean variety is JACK.

8. A breeding method of soybean, comprising overexpressing the gene GmAK-HSDH according to claim 3 in the soybean.

9. The breeding method according to claim 8, comprising introducing the gene GmAK-HSDH into a soybean plant.

10. The breeding method according to claim 8, wherein the soybean variety is JACK.

11. The method according to claim 6, wherein the soybean variety is JACK.

12. The breeding method according to claim 9, wherein the soybean variety is JACK.
